(19) United States
(12) Patent Application Publication
(10) Pub. No.: US 2024/0148545 A1
(43) Pub. Date: May 9, 2024

(54) BOO BOO PAK

(71) Applicants: Karen Jean Waltemire, Hubbard, OH (US); Heather Blake Fumerola, Hubbard, OH (US)

(72) Inventors: Karen Jean Waltemire, Hubbard, OH (US); Heather Blake Fumerola, Hubbard, OH (US)

(21) Appl. No.: 17/981,413

(22) Filed: Nov. 5, 2022

(51) Int. Cl. A61F 7/02

(52) U.S. Cl. CPC A61F 7/02; A61F 2007/0098

(57) ABSTRACT

These therapeutic soft, pliable hot/cold rice packs, in different sizes and shapes, have adjustable elastic strips for the aid in comforting animals recovering from dental surgery and many other injured body parts. They can be frozen, cooled or heated in a microwave. The inner rice bag is hand washable, while the outer fleece covering, having hook and loop closures, can be machine washed and air dried. One or more elastic strips are pulled between silicone/rubber adjuster buttons (which slide independently of each other) and sewn into the product according to size to measure a perfect fit around the injured area.

Hook and loop material could also be sewn onto the bottom elastic strips and bottom of the outer covering for a break-away option.

### FIELD OF INVENTION

This invention, with uncooked natural white rice as a medium, relates to therapeutic hot and cold packs targeted toward the care of injured animals, specifically, but not limited to, after dental surgery. Using the adjustable, thin elastic strips, these rice packs, in different sizes, and shapes can be manipulated to suit an animals' many specific injuries on many body parts. Children and adults can use them in the same manner.

How ours is different-Although there are companies that make rice packs for humans, ours is targeted toward the care of injured animals, specifically for after dental surgery. Using the adjustable, thin elastic strips, these rice packs, in different sizes, and shapes can be manipulated to suit an animals' many specific injuries on many body parts.

For example, (see FIGS. 20, 21), it can be heated for sore muscles on a leg #43 or face #46 or hip #41. Or for arthritis.

It can be chilled to relieve pain and swelling from dental surgery to provide stable soft comfort for the pet. (see FIGS. 19, 22) Also, bumps, bruises and inflammation can be cooled. (see FIGS. 20, 21).

Using hook and loop strips on one end for closing and opening, the outer covering can be machine washed and air dried any time.

The inner bag, using cotton backed soft pliable water-resistant material (as in a restaurant tablecloth) filled with natural white rice, and an outside soft anti-pill fleece (or other soft material for comfort) these packs are created to cover a multitude of pet injuries with silicone/rubber adjuster buttons on elastic strips so that they can be properly and securely adjusted.

### BACKGROUND INFORMATION

People have tried using rice packs on their pets but either had to hold them on for the pet or strap them to secure them. Strapping rice packs to a pet is dangerous. Holding them on is time consuming.

Many herbal packs can be harmful/toxic to cats, dogs, horses, etc. as per the USPCA website. Essential oils can be harmful/toxic to cats, dogs, horses, etc. as per the USPCA website. Among those are common smells that humans love to use: lavender, lemon, mint, orange, aloe, buckwheat, and more can cause diarrhea and/or vomiting in a pet. Ingestion can be fatal if consumed in large quantities.

Synthetic gel packs could be dangerous if punctured.

Antifreeze or ethylene glycol is lethal and has been known to be used in gel packs. Packs using acrylic acid as a thickener can cause rashes.

Although Amorphous Silica compact gel beads are not harmful to pets or humans if ingested, they are not biodegradable nor are they recyclable, as with packs using hydroxyethyl cellulose are not biodegradable.

Hot/cold packs using straps without the elasticity can cause harm to a pet. Ice in a bag is messy and needs a towel or such wrapped around it. Hot water bottles also need protection over it so as not to burn the pet.

### SUMMARY OF INVENTION

Perfect for veterinary use and home use, these form fitting, soft, pliable hot/cold therapy rice packs invention can be heated in a microwave or cooled in a freezer or fridge and are machine washable.

These rice packs are made to be adjusted in different ways for different areas, making it easier on the human as they can guide the pack where it needs to be and let the pet rest without disturbing it, and it permits the animal to have better mobility or relax in its own space

The rubber/silicone buttons can be slid up and down the elastic strips in either direction, independent of each other, to aide in the conformance of the invention to the specific area of injury of the pet.

The outer covering is made of anti-pill fleece (or other soft material) for comfort and is removable for washing and air-drying purposes.

The inner bags are made of cotton lined soft pliable water-resistant plastic (like a restaurant tablecloth) filled with natural white rice according to their size and stitched. The goal is to keep the rice in all areas of the bag for optimal coverage, so the larger packs have stitching in the center.

Although there are companies that make rice packs for humans, ours is targeted toward the care of injured animals, using soft, form-fitting, washable materials having circumference coverage ranges from 7″-35″.

In this invention, the uncooked natural white rice and the anti-pill fleece are biodegradable while the elastic strips, buttons and inner plastic filling material are recyclable.

This invention can also utilize hook and loop material on the bottom of the elastic strips to the bottom of the fleece for a break-away option.

### BRIEF DESCRIPTION OF DRAWINGS

FIGS. 1, 2, 3, and 4 demonstrate how to get the rubber buttons onto the elastic strips using a pair of pliers (#2, 3, 4, 5, and 23)

FIGS. 5, 6, and 7 show how to sew, add rice and complete the bag of rice for the small pack (#6, 7, 8, 9 and 10)

When making the small pack, as in FIG. 5, uncooked natural white rice is added to a pliable cotton backed water-resistant bag that was folded in half then sewn shut.

As in FIG. 8, anti-pill fleece (or other soft material) (#11) has a hook and loop strip sewn into the top (#12) and elastic strips sewn to the outer edges (#13, 14, 15 and 16).

FIGS. 9, 10, 11 and 12 show the process of sewing it shut and adding the rice bag (#22).

As seen in FIGS. 15, 16, 17 and 18, the medium, large, extra-large and the mega pack drawings show the fleece being built in stages of sewing: the hook and loop (#12), the elastic strips (#31, 32 and 33), how to stretch sew the elastic strips (#34, 35 and 36), inserting the rice bag (#28), and where to put the rubber buttons for adjustment.

As with the medium, large, and extra-large packs, the directions for making the water-resistant rice bags are FIGS. 13, 14A and 14B, with 14B being the completed side view.

FIGS. 23, and 24 shows how to get the larger rubber buttons onto the larger elastic strips for the mega pack using a clay molding tool (#47 and 48)

As seen in FIGS. 25, 26 and 27, the mega pack drawings show how to sew in the stitching for the rice to have 3 pockets to sit in. (#51 and 53)

FIGS. 19, 20, 21 and 22 depict the different areas that the hot/cold rice packs can be placed on animals

### DETAILED DESCRIPTION

1. SMALL PACK
Instructions for Creation
2 pieces ¼″ elastic strips @ 7″ each
1 piece small silicone or rubber elastic adjuster bead or tube
1 piece water-resistant plastic tablecloth material with cotton backing @ 6½″ square (or other kind of water-resistant material with pliable plastic)
1 piece anti-pill fleece @ 7″ square or other soft material
2 pieces ⅜″ hook and loop strips@ 3″ each uncooked
natural white rice@3.7 oz.
thread and sewing machine

### Assembly

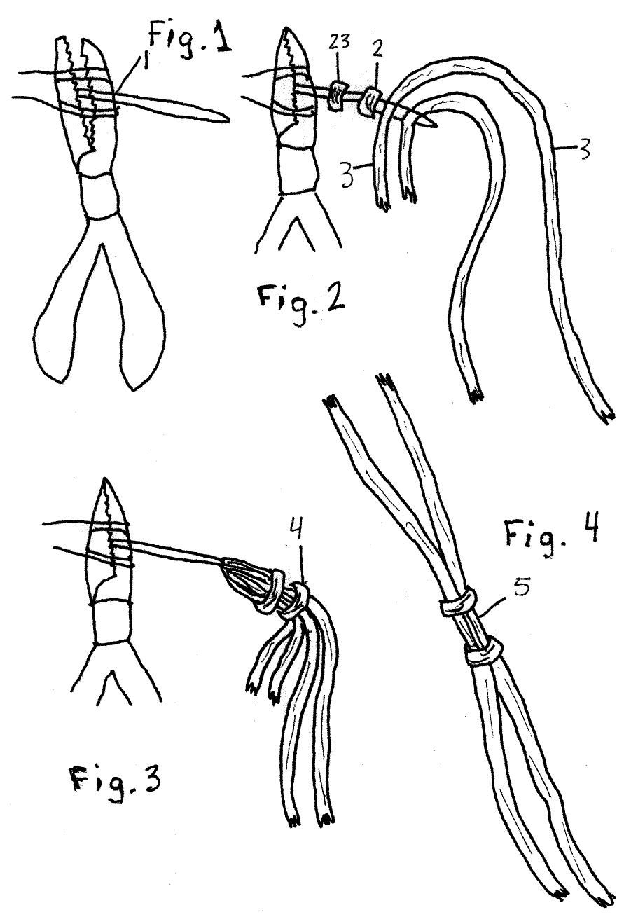

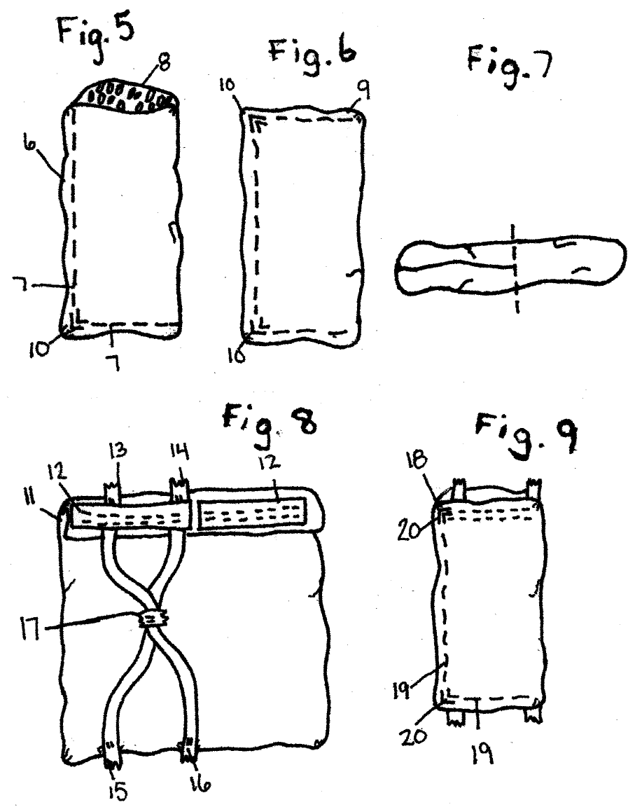

### A. ELASTIC EARS (See FIGS. 1, 2, 3 and 4)

1. Take an 8″ piece of thin wire and fold it in half, leaving a loop.
2. Wrap the wire around a pair of pliers 2 times and hold. #1
3. Slide the two open ends of the wire 2½″ through one rubber button (fat end first). #2
4. Put the 2 elastic strips together and guide 1½″ of length through the loop end of the wire. #3
5. Tighten the pliers and pull the rubber button down until it is in the center of the two straps. #4
6. Remove wire. Ear straps complete. #5

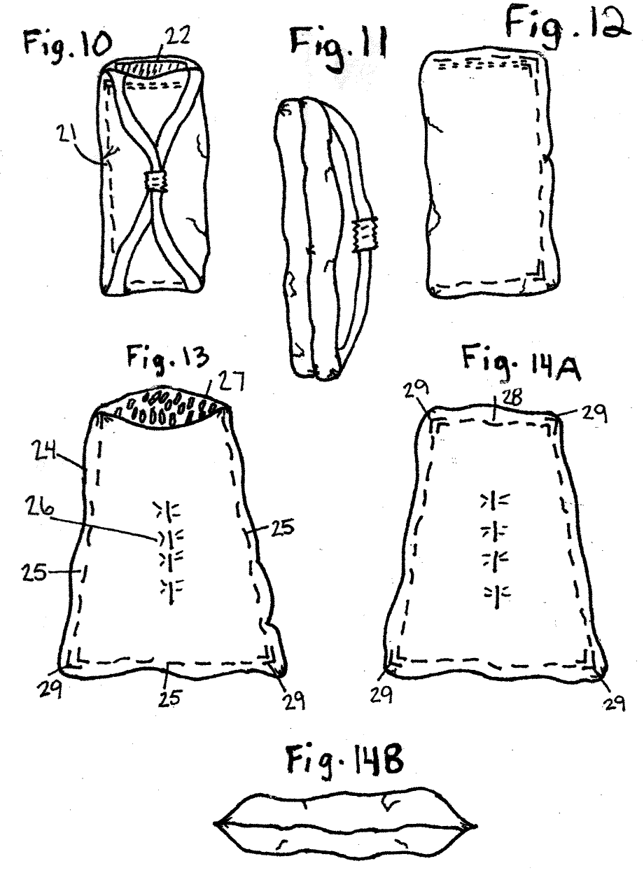

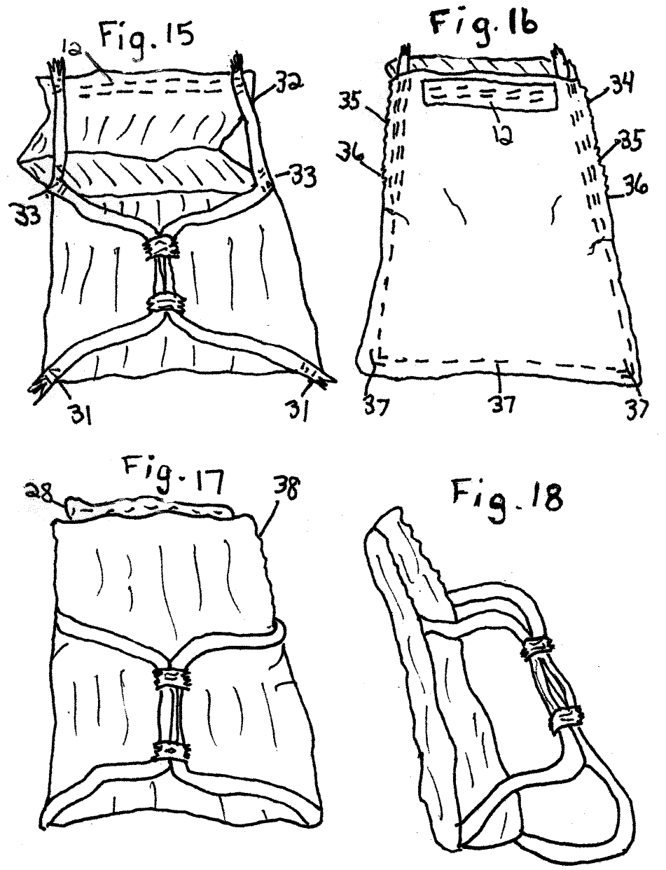

### B. RICE BAG (see FIGS. 5, 6, and 7)

1. Take the 6½″ water-resistant tablecloth material and fold in half plastic side out. #6
2. Sew one short edge shut and the long edge shut. #7
3. Add 3.7 oz. of rice. #8
4. Sew the last edge shut. #9
5. Double stitch all corners. #10

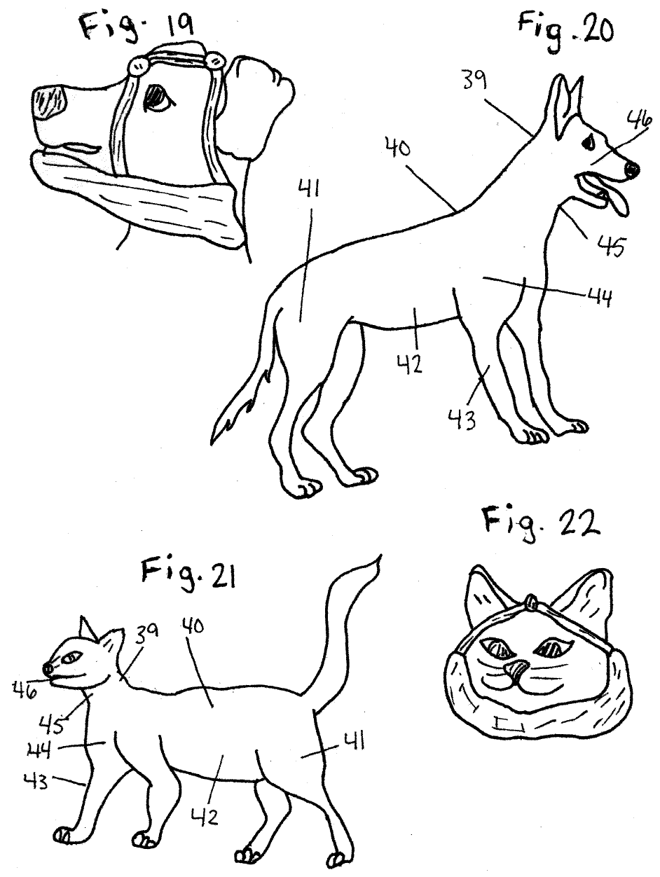

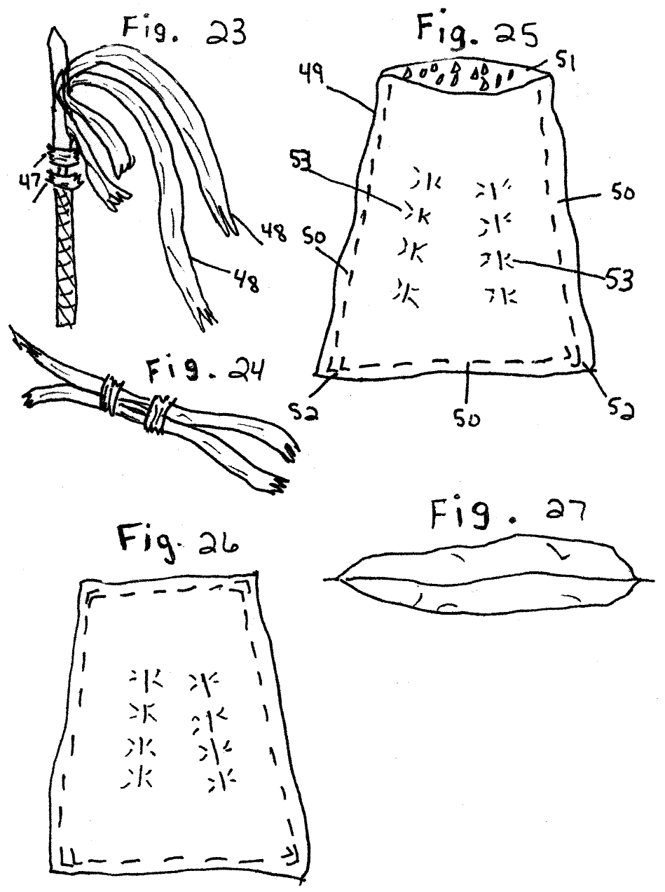

### C. FLEECE (see FIGS. 8, 9, 10, 11 and 12)

1. Take the 7″ fleece and place the sharper image face up on table. #11
2. Fold the top under a ½″.
3. Put the hook and loop strips side by side lining the top of the fleece, one facing up and one facing down. Leave &A″ gap between the two. #12
4. Sew the hook and loop on.
5. Measure a mark at 1″ in from the left edge of top of the fleece, #13, then another mark 2½″ from there #14 (second mark should be in the center).
6. Measure a mark at 1″ in from the left edge of the bottom #15, then another mark 2Y2″ from there #16, (second mark should be in the center).
7. Position the elastic strips so that rubber button is in middle of the marks. #17
8. At the top first mark, stitch the end of an elastic strip. #13
9. Now, follow corresponding end of strip to second mark on bottom and stitch it. #16
10. Now, stitch the end of the other elastic strip to the top second mark. #14
11. Stitch bottom of strip to the first mark on the bottom. #15 (Pattern should resemble an “X”)
12. Now, fold fleece in half bringing the right top corner to the left top comer. #18
13. Sew one short end of fleece closed (double stitching the elastic) and sew the long edge. #19
14. Double stitch all corners. #20
15. Flip right side out. #11
16. Stuff the rice bag inside the fleece. #22
17. Press to close the hook and loop.
2. MEDIUM PACK
2 pieces ¼″ elastic strips @ 12″ each
2 pieces small silicone/rubber elastic adjuster buttons
2 pieces anti-pill fleece (or other soft material):
   width top: 4½″
   width bottom: 6″
   length: 6¾″
2 pieces water-resistant plastic tablecloth material with cotton backing (or other kind of water-resistant material with pliable plastic):
   width top: 3¾″
   width bottom: 4⅞″
   length: 5¾″
2 pieces hook and loop strips @3¾″ long
uncooked natural white rice @ 3.2 oz.
thread and sewing machine

### Assembly

### A. ELASTIC EARS (See FIGS. 1, 2, 3 and 4)

1. Take an 8″ piece of thin wire and fold it in half, leaving a loop.
2. Wrap the wire around a pair of pliers 2 times and hold. #1
3. Slide the two open ends of the wire 2½″ through two rubber buttons (fat end first). #2, #23
4. Put the 2 elastic straps together and guide 2″ of length through the loop end of the wire. #3
5. Tighten the pliers and pull the rubber buttons down until it is in the center of the two straps. #4
6. Remove wire. Ear straps complete. #5

### B. RICE BAG (see FIGS. 13, 14A and 14B)

1. Put two water-resistant tablecloth materials together plastic side facing out. #24
2. Sew three edges shut leaving the shortest edge open. #25
3. Stitch 1Y2″ vertical seam in center of fabric. #26
4. Add 3.2 oz. of rice, half on each side. #27
5. Sew the last edge shut. #28
6. Double stitch all corners. #29

### C. FLEECE (see FIGS. 15, 16, 17 and 18)

1. Take both fleece and place the sharper images face up on table, bottom width on bottom.
2. Fold the top edge under a ½″.
3. Take the two hook and loop strips and place them one on top of each fold, one facing up and one facing down leaving a ¼″ gap on each outside.
4. Sew the hook and loop to fleece. #30
5. Take one fleece and place the sharper image face up on table, bottom width on the bottom.
6. Take the end of an elastic strip and stitch it to the bottom left corner of fleece. #31
7. Follow corresponding end of strip and stitch it to ½″ down from the top right length corner. #32
8. From top right side corner length stitch, measure 2″ down on fleece, measure 1″ down on elastic and stitch together. #33 Stretch the elastic but not the fleece as you sew.
9. Repeat steps 5-8 with the elastic on the opposite sides
10. Place second fleece on top of first, sharper image face down, in mirror image. #34
11. Starting on the short end, begin sewing down the length, tightening the elastic and the fleece as you go. #35
12. Triple sew the stretchy part so that it is invisible within the seams. #36
13. Finish sewing the length down.
14. Sew the bottom width now. #37
15. Sew toward the top, stretching the fleece and elastic as you go. #35
16. Triple sew the stretched hem part so that it is invisible within the seams. #36
17. Double stitch all corners. #37
18. Flip right side out. #38
19. Stuff the rice bag (#48) into the fleece. #38
20. Press the hook and loop strips closed.

### 3. LARGE PACK and EXTRA-LARGE PACK

2 pieces ¼″ or ½″ elastic strips @ 13½″ each FOR LARGE
2 pieces ¼″ or ½″ elastic strips @ 15½″ each FOR EXTRA-LARGE
2 pieces small silicone/rubber elastic adjuster buttons for the ¼″ or medium silicone/rubber buttons for the ½″ strips
2 pieces anti-pill fleece (or other soft material):
   width top: 5½″
   width bottom: 7⅛″
   length: 8¼″
2 pieces water-resistant plastic tablecloth material with cotton backing, (or other kind of water-resistant material with pliable plastic):
   width top: 5″
   width bottom: 6¾″
   length: 7⅞″
2 pieces hook and loop strips @ 5″ long
uncooked natural white rice @ 7 oz.
thread and sewing machine

### Assembly

### A. ELASTIC EARS (See FIGS. 1, 2, 3 and 4)

1. Take an 8″ piece of thin wire and fold it in half, leaving a loop.
2. Wrap the wire around a pair of pliers 2 times and hold. #1
3. Slide the two open ends of the wire 2½″ through two rubber buttons (fat end first). #2, #23
4. Put the 2 elastic strips together and guide 2″ of length through the loop end of the wire. #3
5. Tighten the pliers and pull the rubber buttons down until it is in the center of the two straps. #4
6. Remove wire. Ear straps complete. #5

### B. RICE BAG (See FIGS. 13, 14A and 14B)

1. Put two water-resistant tablecloth materials together plastic side facing out. #24
2. Sew three edges shut leaving the shortest edge open. #25
3. Stitch 2″ vertical seam in center of fabric. #26
4. Add 7 oz. of rice, half on each side. #27
5. Sew the last edge shut. #28
6. Double stitch all corners #29

### C. FLEECE (See FIGS. 15, 16, 17 and 18)

1. Take both fleece and place the sharper images face up on table, bottom width on bottom.
2. Fold the top edge under a ½″.
3. Take the two hook and loop strips and place them one on top of each fold, one facing up and one facing down leaving a ¼″ gap on each outside.
4. Sew the hook and loop to fleece. #30
5. Take one fleece and place the sharper image face up on table, bottom width on the bottom.
6. Take the end of an elastic strip and stitch it to the bottom left corner of fleece. #31
7. Follow corresponding end of strip and stitch it to Y2″ down from the top right length comer. #32
8. From top right side corner length stitch, measure 2″ down on fleece, measure I″ down on elastic and stitch together. #33. Stretch the elastic but not the fleece as you sew.
9. Repeat steps 5-8 with the elastic on the opposite sides
10. Place second fleece on top of first, sharper image face down, in mirror image. #34
11. Starting on the short end, begin sewing down the length, tightening the elastic and the fleece as you go. #35
12. Triple sew the stretchy part so that it is invisible within the seams. #36
13. Finishing sewing the length down.
14. Sew the bottom width now. #37
15. Sew toward the top, stretching the fleece and elastic as you go. #35
16. Triple sew the stretched hem part so that it is invisible within the seams. #36
17. Double stitch all corners. #37
18. Flip right side out. #38
19. Stuff the rice bag (#28) into the fleece. #38
20. Press the hook and loop strips closed.

### Megapack

2 pieces ½″ elastic strips @ 21″ each
2 pieces large silicone/rubber elastic adjuster buttons
2 pieces anti-pill fleece (or other soft material):
   width top: 7″
   width bottom: 9″
   length: 8¼″
2 pieces water-resistant plastic tablecloth material with cotton backing, (or other kind of water-resistant material with pliable plastic):
   width top: 6½″
   width bottom: 8½″
   length: 7⅞″
2 pieces hook and loop strips @ 6″ long
uncooked natural white rice @ 10 oz.
thread and sewing machine

### Assembly

### A. ELASTIC EARS (See FIGS. 23 and 24)

1. Take a clay molding tool with a loop at the end.
2. Shove 2 large rubber buttons down the loop end of the tool. #47
3. Put two ⅜″ elastic strips together and guide them 1″ through the loop. #48
4. Pull the rubber buttons down the elastic strips.
5. Remove elastic from clay molding tool. Ear straps complete.

### B. RICE BAG (See FIGS. 25, 26, and 27)

1. Put two water-resistant tablecloth materials together, plastic side facing out. #24
2. Sew three edges shut leaving the shortest edge open. #50
3. Stitch 2″ vertical seam at ⅓″ and ⅔″ of fabric making three (3) pockets at 1½″ down. #53
4. Add 10 oz. of rice, one third on each third of pockets. #51
5. Sew the last edge shut.
6. Double stitch all comers #52

### C. FLEECE (See FIGS. 15, 16, 17 and 18)

1. Take both fleece and place the sharper images face up on table, bottom width on bottom.
2. Fold the top edge under a ½″.
3. Take the two hook and loop strips and place them one on top of each fold, one facing up and one facing down leaving a ½″ gap on each outside.
4. Sew the hook and loop to fleece. #30
5. Take one fleece and place the sharper image face up on table, bottom width on the bottom.
6. Take the end of an elastic strip and stitch it to the bottom left corner of fleece. #31
7. Follow corresponding end of strip and stitch it to ½″ down from the top right length comer. #32
8. From top right side corner length stitch, measure 2″ down on fleece, measure 1″ down on elastic and stitch together. #33. Stretch the elastic but not the fleece as you sew.
9. Repeat steps 5-8 with the elastic on the opposite sides.
10. Place second fleece on top of first, sharper image face down, in mirror image. #34
11. Starting on the short end, begin sewing down the length, tightening the elastic and the fleece as you go. #35
12. Triple sew the stretchy part so that it is invisible within the seams. #36
13. Finish sewing the length down.
14. Sew the bottom width now. #37
15. Sew toward the top, stretching the fleece and elastic as you go. #35
16. Triple sew the stretched hem part so that it is invisible within the seams. #36
17. Double stitch all corners. #37
18. Flip right side out. #38
19. Stuff the rice bag (#49) into the fleece. #38
20. Press the hook and loop strips closed.

### Usage Examples

Perfect for veterinary care and home care, being used as needed as a hot pack or cold pack, these soft, pliable rice packs, made in different sizes (and can also be created in different shapes according to the animal's anatomy) will aid in the healing of a pet's jaw, head, chin, neck, leg, hip, ankle, back, belly, wherever needed.

For children and adults, this invention can help alleviate pain or swelling in an arm, leg, head, chin, cheek, neck, foot, ankle, hand, etc.

This invention can also utilize hook and loop material on the bottom of the elastic strips to the bottom of the fleece for a break-away option.

1. These therapeutic hot/cold rice packs, in different sizes, designed mainly for use for veterinarians, on various animal (or human) injuries, can be placed in the fridge, freezer and microwave, comprising:
in this instance, a soft outside covering, hook and loop closures on the top for easy removal/replacement of the pliable water-resistant inner bag, filled with rice and sewn shut that will comfortably conform around an animals' injury with a plurality, in this instance, two (2) soft elastic strips butted through one or more independently adjustable rubber buttons for a snug fit.

2. As in claim 1, the inner bag of the small pack is one square piece of cotton backed soft pliable water-resistant material (as in a restaurant tablecloth) filled with uncooked natural white rice and sewn completely shut.

3. As in claim 1, the inner bags of the medium, large, and extra-large packs, in this instance, are two (2) cotton backed soft pliable water-resistant material (as in a restaurant tablecloth) sewn together with stitching in the center to aide in the proper disbursement of the uncooked natural white rice through the entire bag.

4. As in claim 1, the inner bag of the mega pack is vertically sewn at ⅓″ and ⅔″ in middle to aide in the proper disbursement of the rice through the entire bag.

5. As in claim 1, the medium used for these inner bags is uncooked natural white rice which is protected from liquid by the bag which is completely sewn shut after filling and are free of essential oils, herbal plants/essences, toxins, and chemicals.

6. As in claim 1, the covering for the small size uses one square piece of anti-pill fleece folded in half with hook and loop on each top half, then two (2) soft elastic strips stitched into each top/bottom left corner, then top/bottom center using, in this instance, one adjuster button.

7. As in claim 1, the outer coverings of the medium, large, and extra-large sizes, in this instance, will use two (2) pieces of anti-pill fleece, hook and loop strips sewn into each top piece then two (2) soft elastic strips sewn directly into the fabric, having three (3) inches down each side (or such) of fleece stretched during the sewing process, making the fabric stretchy, conforming gently at the front end of the pack then stitched into the bottom corners for stability and adjustability of, for this application, two (2) adjuster buttons which slide independently of each other for a perfect snug fit to the injury.

8. As in claim 1, the outer covering of the mega pack, in this instance, uses larger ⅜″ elastic strips with larger adjuster buttons for more durability for the heavier inner rice pack that is inside of it.

9. As in claim 1, the packs in their entirety (of all sizes) can be frozen for any length of time or refrigerated for any length of time and placed directly on an injury, beings that the outer covering is of anti-pill fleece, which protects the user from being burned by the sub-zero temperature.

10. As in claim 1, the small and medium sized packs, being microwaved for approximately 25 seconds, and the large and extra-large packs microwaved for approximately 45 seconds and the mega pack microwaved for approximately 60 seconds make for ideal therapeutic temperature:
the difference being the amount of rice inside the inner bag.

11. As in claim 1, the outer fleece covering form-fits around the inner rice bag to cover the entire surface area of the completed pack for optimal hot/cold coverage.

12. As in claim 1, the outer fleece covering is removable for machine washing and air-drying whilst the inner rice bag is hand wash only as not to moisten the rice inside of it.
